(19) United States
(12) Patent Application Publication
(10) Pub. No.: US 2015/0224286 A1
(43) Pub. Date: Aug. 13, 2015

(54) CATHETER DEVICES WITH SECURING MECHANISMS AND RELATED METHODS

(71) Applicant: B. BRAUN MELSUNGEN AG, Melsungen (DE)

(72) Inventors: E-Jen Teh, Penang (MY); Hwa Loon Chan, Penang (MY)

(21) Appl. No.: 14/616,440

(22) Filed: Feb. 6, 2015

Related U.S. Application Data
(60) Provisional application No. 61/937,242, filed on Feb. 7, 2014.

(51) Int. Cl. A61M 25/02; B32B 37/18; B32B 38/10; B32B 37/12; B32B 37/26; B32B 38/00; A61M 25/00; B32B 37/14

(52) U.S. Cl. CPC A61M 25/02; A61M 25/0014; B32B 37/18; B32B 37/142; B32B 37/12; B32B 37/26; B32B 38/0012; B32B 38/10; A61M 2025/0253; B32B 2037/268; B32B 2535/00

(57) ABSTRACT

Catheter securement devices are generally described for securing the catheter hub to the access site following successful catheterization. Examples of catheter assemblies with integrated securing mechanisms and related methods are disclosed. The integrated device provides convenience and easy to use access to securing devices that are integrated to the wings of the catheter assemblies.

### FIELD OF ART

The present disclosure generally relates to catheter devices and more particularly to catheter devices having securing mechanisms and related methods.

### BACKGROUND

After successful catheterization of an indwelling catheter to a patient, the access site where the catheter tube enters the skin is secured to prevent the catheter from moving or inadvertently withdrawing from the vessel of the patient. To secure the catheter to the patient, a practitioner typically uses tape or other catheter stabilization devices. These catheter stabilization devices are usually separate products that come in their own packaging.

### SUMMARY

The various embodiments of a catheter device having a securing mechanism have several features, no single one of which is solely responsible for their desirable attributes. Without limiting the scope of the present embodiments as set forth in the claims that follow, their more prominent features now will be discussed briefly.

Aspects of the present disclosure include a catheter assembly with securing mechanisms which includes a catheter hub unit comprising a catheter hub, a catheter tube extending distally from the catheter hub, and two wings each extending laterally of the catheter hub, a needle hub unit comprising a needle hub and a needle extending distally from the catheter hub and projecting through the catheter tube in a ready position, an anchor sheet located on each of the wings, each anchor sheet comprising a top side facing away from a corresponding wing and a bottom side facing the corresponding wing in the ready position, an adhesive layer provided on the top side of each anchor sheet, and a release liner covering the adhesive layer on each anchor sheet in the ready position. Each anchor sheet can be pivotably connected to the corresponding wing.

Each anchor sheet can be pivotably connected to the corresponding wing in a different direction from each other.

Each wing can be shaped differently from the other wing. A recessed space can be formed in each wing, the recessed space being configured for securing the anchor sheet in the ready position.

A tab can extend from each anchor sheet, the tab being configured for pulling the anchor sheet from the recessed space.

A release liner tab can extend from the release liner.

Each anchor sheet can be pivotably connected to the respective wing by adhesive along a pivoting edge on the anchor sheet. The pivoting edge for each wing can differ in location from the other or be the same.

An anchor sheet can be provided on a bottom exterior surface of the catheter hub in addition to the anchor sheets on the wings.

Each anchor sheet can be pivotably connected to the respective wing by a living hinge.

The living hinge can be located at a proximal end of the wing.

The release liner can be attached to the needle hub unit, such that withdrawing the needle hub unit from the catheter hub unit rotates the anchor sheet and removes the release liner.

The anchor sheets can include a plurality of connected subsections folded upon each other in an accordion fashion to form folded anchor sheets.

The needle hub unit can further comprise a pair of tethers attached to a free end of the folded anchor sheets.

The folded anchor sheets can be stored in a storage space in the wing. The folded anchor sheets can be tucked under the wing. The folded anchor sheets can unfold to extend in a same first direction.

One of the two folded anchor sheets can unfold to extend in a distal direction with the adhesive facing down, and the other of the two anchor sheets can unfold to extend in a proximal direction with the adhesive facing down.

Because the anchor sheets are accessible once applied to a patient, they can easily be lifted and removed to facilitate removing the catheter hub from the patient. For example, the anchor sheets can be lifted directly using fingers or other means to remove the catheter hub from the patient. Anchor sheets that are not directly accessible, such as being located only on lower surfaces of the wings or the catheter hub, can be difficult to remove.

Another aspect of the present disclosure includes a method of manufacturing a catheter assembly which includes extending a catheter tube distally from a catheter hub, a left wing laterally from a left side of the catheter hub, and a right wing laterally from a right side of the catheter hub, extending a needle from a needle hub unit through the catheter tube in a ready position, pivotably connecting an anchor sheet to the left wing and another anchor sheet to the right wing, each anchor sheet comprising a top side facing away from a corresponding wing and a bottom side facing the corresponding wing in the ready position, the top side having an adhesive layer, and covering the adhesive layer on each anchor sheet with a release liner.

Each anchor sheet can be pivotably connected to the corresponding wing in a different direction from each other.

A recessed space can be formed in each wing, the recessed space securing the anchor sheet in the ready position.

The method can further include pulling the anchor sheet from the recessed space.

Each anchor sheet can be pivotably connected to the corresponding wing by a living hinge.

The living hinge can be located at a proximal end of the wing.

The method can further include withdrawing the needle hub from the catheter hub thereby rotating the anchor sheet and removing the release liner, wherein the release liner is attached to the needle hub.

The method can further include folding the anchor sheets into a plurality of connected subsections folded upon each other in an accordion fashion.

The method can further include unfolding the anchor sheets by pulling a free end of the folded anchor sheets with tethers attached to the needle hub.

The method can further include storing the anchor sheets in a storage space in the wing.

The method can further include tucking the folded anchor sheets under the wing.

The method can further include folding the anchor sheets over the wings towards a distal direction after unfolding the anchor sheets.

The method can further include unfolding the anchor sheets in different directions.

The method can further comprise providing direct access to the anchor sheets so that they can be gripped, such as by an instrument or by fingers.

The method can further comprise the step of applying the anchor sheets to a patient.

The method can further comprise the step of directly gripping the anchor sheets and lifting the anchor sheets away from the patient.

Each anchor sheet can be removed from the skin of the patient with or without tabs for easy removal of the catheter.

### DESCRIPTION OF DRAWINGS

These and other features and advantages of the present device, system, and method will become appreciated as the same becomes better understood with reference to the specification, claims and appended drawings wherein:

FIG. 1 shows one embodiment of a catheter assembly, the catheter assembly including a catheter hub unit and a needle hub unit;

FIG. 2 shows the catheter assembly of FIG. 1 with release liners of the catheter hub unit being removed;

FIG. 3 shows the catheter assembly of FIG. 1 with anchor sheets of the catheter hub unit pivoted from the wings;

FIG. 4 shows another embodiment of a catheter assembly;

FIG. 5 shows an enlarged view of the circular portion 5-5 of FIG. 4;

FIGS. 6-9 show various stages of separating a needle hub unit from a catheter hub unit of yet another embodiment of a catheter assembly, the catheter hub unit including wings and anchor sheets attached to the wings, and the needle assembly tethered to the anchor sheets;

FIG. 10 shows one embodiment of wings of the catheter assembly of FIG. 6;

FIG. 11 shows another embodiment of wings of the catheter assembly of FIG. 6; and

FIG. 12 shows a front view of the catheter assembly of FIG. 11.

### DETAILED DESCRIPTION

The detailed description set forth below in connection with the appended drawings is intended as a description of the presently preferred embodiments of catheter assemblies for use in various applications provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

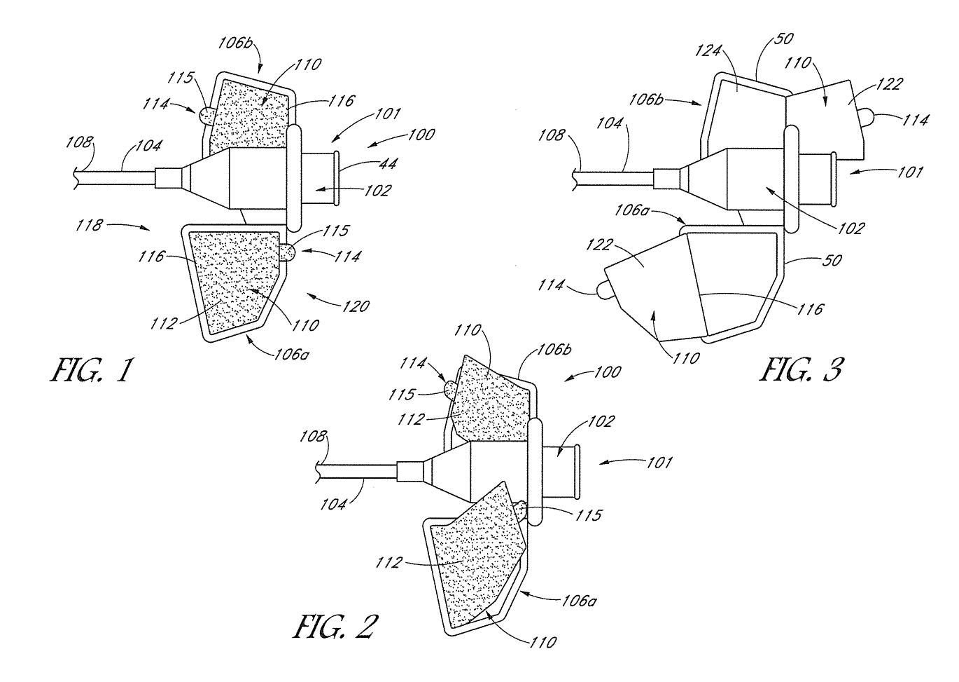

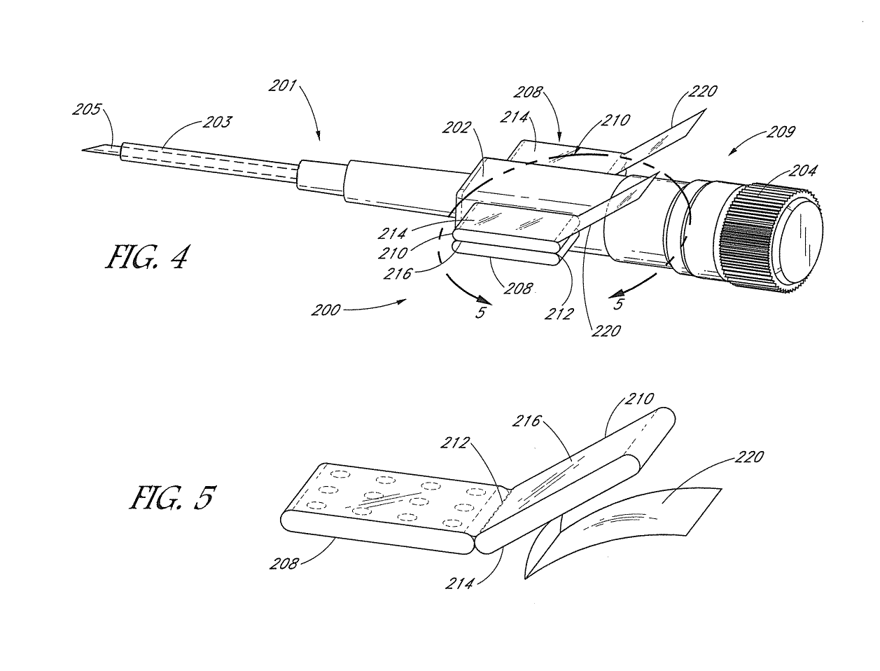

FIG. 1 illustrates one embodiment of a catheter assembly 100, which includes a catheter hub unit 101 and a needle hub unit (conventional and shown in FIG. 4). The catheter hub unit 101 includes a catheter hub 102, a catheter tube 104 connected to a distal end of the catheter hub 102, and a pair of wings 106a, 106b extending laterally from the catheter hub 102. For example, a first or left wing 106a extends from a left side of the catheter hub 102, and a second or right wing 106b extends from a right side of the catheter hub 102.

A proximal end opening 44 of the catheter hub 102 may include a female Luer fitting (not shown) having a female Luer taper and/or female Luer lock threads. The female Luer fitting is thus configured to matingly receive a male Luer connector, such as used in connection with an IV line, a Luer access connector, a needle hub, a syringe tip, a vent plug, or another known or future-developed IV devices. Each of these components can be sized and configured in conformity with at least some of the International Standards Organization (ISO) standards for female and male Luer connections under current or future standards.

The catheter tube 104 is shown penetrated under the skin into the vein of a patient at an access site 108. Typically, a needle tip of a needle extends through a hollow interior cavity of the catheter hub 102 from a distal end of the needle hub and out the distal opening of the catheter tube 104, prior to insertion into the patient at the access site 108. The needle tip can also extend through various components such as a valve, a valve actuator, and a needle guard, positioned in the hollow interior cavity of the catheter hub 102, the examples of which can be found in U.S. Pat. No. 8,333,735, the contents of which are expressly incorporated herein by reference. The needle guard may also be located outside of the catheter hub as disclosed in U.S. Pat. No. 8,597,249, the contents of which are expressly incorporated herein by reference.

A distal portion of the catheter tube 140 can taper inwardly or the distal opening can have a size smaller than an outer diameter of the needle to form a seal around the opening of the catheter tube 103 with the needle to prevent fluid from entering the annular space between the catheter tube 102 and the needle when the needle tip pierces the skin of the patient. After verification of proper needle and catheter tube placement, retraction of the needle tip in a proximal direction will allow fluid or blood to flow into the annular space between the needle and the interior of the catheter tube 102, known as secondary blood flashback. After the needle tip and distal end of the catheter tube 104 has penetrated and accessed the vein, the needle hub is then withdrawn from the catheter hub unit 101 to remove the needle from the catheter hub 102. When incorporated, a needle guard will activate to cover the needle tip to prevent inadvertent needle stick.

In the example shown, an anchor sheet 110 is provided on each wing 106a, 106b. The anchor sheet 110 is configured to secure the catheter hub unit 101 to the patient and prevent the catheter hub unit 101, including the catheter tube 104, from shifting in and out, or around the access site 108, where the catheter tube penetrates the skin. Each anchor sheet 110 has a top side and an opposed bottom side facing the respective wing 106a, 106b. The top side of each anchor sheet 110 is provided with an adhesive, such as a hypoallergenic adhesive or a pressure-sensitive adhesive, and a release liner 112 for covering the adhesive until use. In one embodiment, each anchor sheet 110 is securely attached to the respective wing 106a, 106b along a pivoting edge 116. In another embodiment, each anchor sheet 110 is securely attached to the respective wing 106a, 106b along a pivoting edge 116 and wedged in a recessed space 124 defined by a perimeter lip 50 of the respective wing 106a, 106b to hold the anchor sheet 110 in place prior to use. The pivoting edge 116 of the two anchor sheets can be located at any edge of the wings 106a, 106b. In one embodiment, the location of the two pivoting edges 116 of the two anchor sheets 110 are reversed. For example, the pivoting edge 116 at the left wing 106a is on the distal side 118 of the left wing 106a while the pivoting edge 116 at the right wing 106b is on the proximal side 120 of the right wing 106b. In another embodiment, the location of the two pivoting edges 116 can also be located on the same side such as the distal side 118 or the proximal side 120 of the wings 106a, 106b. In yet another embodiment, the location of the two pivoting edges 116 are at the edges furthest away from the catheter hub 102. The wings 106a, 106b can be symmetrically identical or different from each other. In the illustrated embodiment, the wings 106a, 106b are shaped such that the pivoting edge 116 is sufficiently long enough to adequately secure the catheter hub unit 101 to the patient and provide a minimum rotation or movement at the pivoting edge 116. A tab 114 extends outwardly from each anchor sheet 110 to allow a user to pull the anchor sheet 110, such as from the recessed space 124 of the respective wing 106a, 106b, or the surface of the respective wings 106a, 106b if no recessed space 124 is provided. In a particular example, each pivoting edge 116 comprises a band of permanently bonded anchor sheet layer, bonded to the surface of the respective wing 106a, 106b, with a folded line along or adjacent the band to form a fold for pivoting.

FIG. 2 shows the release liner 112 being removed from the top side of each anchor sheet 110 and the anchor sheet 110 lifted from the recessed space 124 of the respective wing 106a, 106b by pulling up on the tab 114 or by removal of the release liner 112 from a different gripping process or location of the release liner. A tab liner 115, which may also be called a release liner tab, can extend from the release liner 112 to help separate the release liner 112 from the anchor sheet 110. For example, if there is no adhesive on the tab 114 and the tab liner 115 overlaps the tab 114 of the anchor sheet, a user can easily grip the tab liner 115 to pull off the release liner 112. No adhesive on the tab 114 also allows a user to easily remove the anchor sheet 110 from the patient when removing the catheter hub unit 101 from the patient, by gripping and pulling the anchor sheet 110 from the tab 114. In another example, the anchor sheet 110 does not have a tab 114 and the anchor sheet 110 is lifted from the recessed space 124 of the respective wing 106a, 106b by pulling on the release liner tab 115 or gripping one of the edges of the anchor sheet.

FIG. 3 shows the two anchor sheets 110 rotated along the pivoting edge 116 so that the top side of each anchor sheet 110, which is provided with adhesive, can be pressed against the skin of the patient to secure the catheter hub unit 101 and the catheter tube 104 to the access site 108. After the release liner 112 is removed from the anchor sheet 110 to expose the adhesive on the top side of the respective anchor sheet 110, the anchor sheet 110 is removably fixed to the skin of the patient by pressing against the bottom side 122 of the anchor sheet 110 to press the now exposed adhesive layer on the top side against the skin of the patient. In some examples, the recessed space 124 of each wing 106a, 106b may be provided with a testing device or instrument, such as a glucose strip, which is only exposed after the anchor sheets 110 are pivoted and bonded to the patient.

FIG. 4 shows another embodiment of a catheter assembly 200 which includes a catheter hub unit 201 and a needle hub unit 209 attached to a proximal end of the catheter hub unit 201. The catheter hub unit 201 includes a catheter hub 202, a catheter tube 203 extending distally from the catheter hub 202, and a pair of wings 208 extending laterally from the catheter hub 202. In the example shown, an anchor sheet 210 is provided with each wing 208 and each anchor sheet 210 has an adhesive layer or coat covered by a release liner 220. The needle hub unit 209 shown includes a needle hub 204 attached to the proximal end of the catheter hub 202, and a needle 205 extending distally from the needle hub 204 and through the catheter hub 202 and the catheter tube 203. The catheter assembly 200 is similar to the catheter assembly 100 of FIG. 1, except that the anchor sheet 210 is integrally formed with the wing 208, as further discussed below.

With reference now to FIG. 5, which is an enlarged view of one of the wings of FIG. 4, the anchor sheet 210 is shown attached to the wing 208 along a hinge 212. In the illustrated embodiment, the hinge 212 is located at a proximal side of the wing 208. However, the location of the hinge 212 is not limited and can be located at any of the various sides of the wing 208. In the example shown, the anchor sheet 210 is unitarily formed with the wing 208 and the hinge 212 is a living hinge. Thus, the anchor sheet 210 is made from the same thermoplastic material as the wing 208. When in the ready to use position as shown in FIG. 4, the anchor sheet 210 has a top side 214 facing away from the wing 208 and a bottom side 216 facing the wing 208. To temporarily secure the anchor sheet 210 to the wing 208 in the ready to use position, detents or a weak adhesive can be used.

Hypoallergenic adhesive or pressure-sensitive adhesive may be provided on the top side 214 of each anchor sheet 210 and is protected by a release liner 220. FIG. 5 shows the anchor sheet 210 pivoted away at the hinge 212 from the wing 208 with the release liner 220 in the process of being removed from the top side 214 of the anchor sheet 210. In one embodiment, the two release liners 220 are secured to the needle hub 204 so that upon retraction of the needle hub unit 209 away from the catheter hub unit 201 following successful catheterization, the anchor sheets 210 are pulled by the retracting needle hub unit 209 and automatically rotated about the hinge 212. Concurrently therewith, the release liners 220 are automatically removed from the anchor sheets 210 thereby exposing the adhesive for attaching to the patient. In another embodiment, the two release liners 220 are not attached to the needle hub unit 209 and can be removed from the anchor sheets 210 manually by the user. Once the release liner 220 is completely removed from the anchor sheet 210, force is applied to the bottom side 216, now rotated to face up with respect to the wing 208 as shown in FIG. 5, to press the adhesive against the skin, thus securing the catheter hub 202 and the catheter tube 203 to secure the access site from unwanted or from excessive movement.

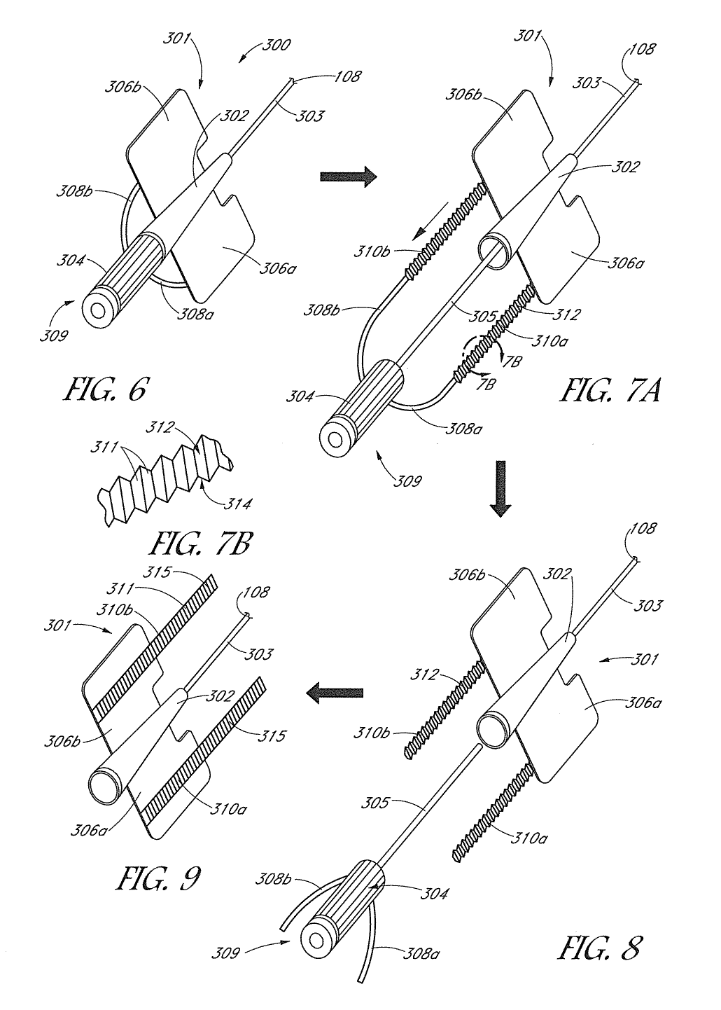

With reference now to FIG. 6, yet another embodiment of a catheter assembly 300 is shown partially inserted into an access site 108. The catheter assembly 300 includes a catheter hub unit 301 and a needle hub unit 309 attached to a proximal end of the catheter hub unit 301. The catheter hub unit 301 includes a catheter hub 302, a catheter tube 303 extending distally from the catheter hub 302, and a pair of wings 306a, 306b extending laterally of the catheter hub 302. An anchor sheet 310a, 310b (FIG. 7A) is provided with each corresponding wing 306a, 306b, and each has an adhesive layer or coat, as further discussed below. The needle hub unit 309 includes a needle hub 304 attached to the proximal end of the catheter hub 202, and a needle 305 with a sharpened distal tip extending distally from the needle hub 304 and through the catheter hub 302 and the catheter tube 303. The catheter assembly 300 is similar to the catheter assembly of FIG. 1 except that the anchor sheets 310a, 310b are folded upon each other in an accordion fashion, with or without a separate release liner. In the illustrated embodiment, a pair of tethers 308a, 308b are connected to the needle hub 304 and to the folded anchor sheets 310a, 310b, and the folded anchor sheets 310a, 310b have ends that are connected to the catheter hub 202. In an alternative embodiment, the tethers 308a, 308b are omitted and the two folded anchors sheets 310a, 310b are to be activated or deployed when a user grips and manipulates them.

FIG. 7A shows the needle hub 304 retracted away from the catheter hub 302, such as following successful catheterization. Referring also to FIG. 7B, the folded anchor sheets 310a, 310b each includes a plurality of connected subsections 311 stacked upon each other. The length and width of the connected subsections 311 can vary depending on the design. In addition, the shape of the connected subsections is not limited to square or rectangular, but can be any regular or irregular shape. In the process of retracting the needle hub unit 309 from the catheter hub unit 301, such as following successful venipuncture, the tethers 308a, 308b, which are attached to the two folded anchors sheets 310a, 310b, pull the two folded anchor sheets 310a, 310b away from the two wings 306a, 306b. When pulled, the folded and connected subsections 311 of the anchor sheets 310a, 310b are unfolded and lengthen. Each unfolded anchor sheet 310a, 310b comprises a top side 312 and a bottom side 315 opposite the top side. The anchor sheets 310a, 310b are provided with hypoallergenic adhesive or pressure-sensitive adhesive on at least some portions or substantially all along the top side 312. In one example, the adhesive is only on the top side 312 of every other connected subsection 311, such that the adhesive will be protected by an adjacent subsection 311 without the adhesive, which acts as a release liner. In another example, the adhesive is on substantially the entire top side 312 of the anchor sheet 310a, 310b. In yet another example, a separate release liner is provided to cover the adhesive on the top side 312 of the anchor sheet 310a, 310b. The connected subsections 311 can be formed by simple folds, double folds, or various fold patterns from an elongated sheet with uniform or non-uniform outer edges to form a sufficiently compact configuration for storage under the wings in a ready to use position.

FIG. 8 shows the needle hub unit 309 completely separated from the catheter hub unit 301. The two tethers 308a, 308b are separated from the two folded anchor sheets 310a, 310b. Perforations or weakened sections may be provided at attachment points between the tethers 308a, 308b and the anchor sheets 310a, 310b to facilitate separation. In one example, the tethers 308a, 308b are lightly attached to at least one connected subsection 311 of the anchor sheets 310a, 310b and detach from the anchor sheets 310a, 310b when the anchor sheets 310a, 310b are fully extended. In another example, if a separate release liner is provided, the release liner is also attached to the tethers 308a, 308b and removed from the anchor sheets 310a, 310b when the tethers 308a, 308b are separated from the two folded anchor sheets 310a, 310b.

Referring to FIG. 9, the two folded anchor sheets 310a, 310b are folded over the two wings 306a, 306b with a plurality of straightened connected subsections 311 extending beyond the wings 306a, 306b. The bottom sides 314 of the anchor sheets 310a, 310b are pressed against the skin to adhere the anchor sheets 310a, 310b to the skin to secure the catheter hub 302 and the catheter tube 303 to the access site 108. In some examples, surfaces on an underside 54 of the wings are also provided with adhesive to further secure the wings to the skin of the patient.

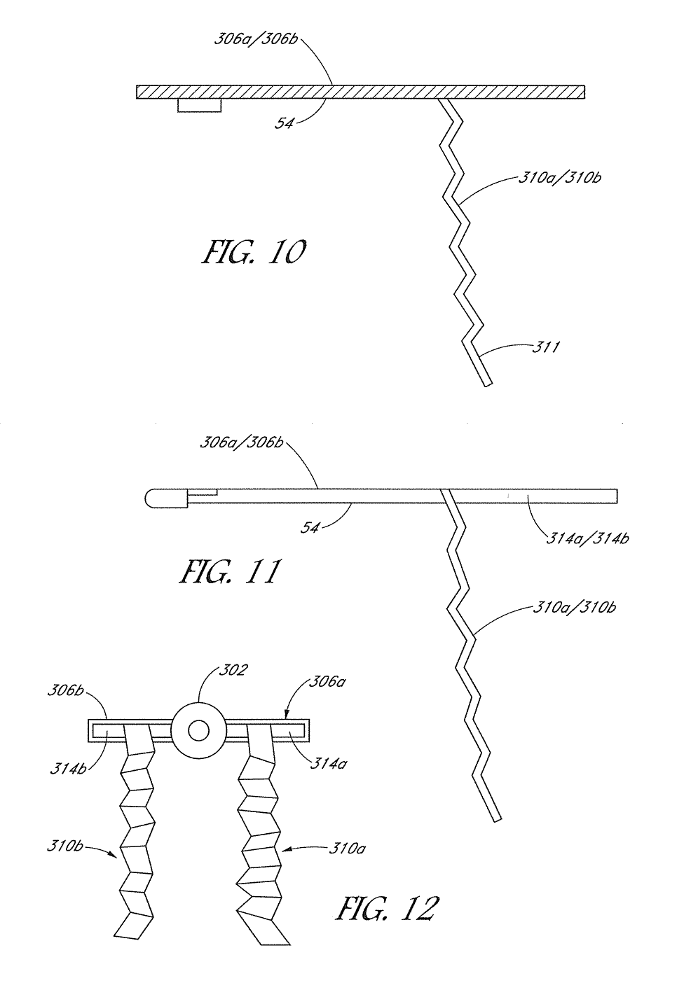

FIG. 10 shows one configuration for storing the folded anchor sheets 310a, 310b underneath the wings 306a, 306b. In the illustrated embodiment, the anchor sheets 310a, 310b are folded and attached to the exterior underside 54 of the wings in the ready to use position of FIG. 6, which is the side that faces the patient when pressed against the patient. Each tether 308a, 308b (not shown) is attached to a respective free end of the anchor sheet 310a, 310b, or possibly even near the respective free end if not right at the free end.

FIG. 11 shows a second configuration for storing the folded anchor sheets 310a, 310b underneath the wings 306a, 306b. In the present embodiment, the anchor sheets 310a, 310b are folded and attached to a hollow space or a storage space 314a, 314b inside each respective wing 310a, 310b. FIG. 12 shows each wing 306a, 306b having the storage space 314a, 314b. The folded anchor sheets 310a, 310b are tucked away inside the storage space 314a, 314b in the ready to use position. The tethers 308a, 308b (not shown) attach to the free ends of the anchor sheets 310a, 310b. The location of the folded anchor sheets 310a, 310b is not limited and can be stored anywhere on the wing such as underneath the wings 306a, 306b as discussed above for FIGS. 6-12 or above the wings 306a, 306b. For example, the anchor sheets 310a, 310b can be folded and attached at a top of the wings in the ready to use position of FIG. 6.

It is also understood that the top or bottom sides 312, 315 of the anchor sheets 310a, 310b having the adhesive could be applied to the skin in any direction such as in a direction towards the access site 108 or away from the access site 108, depending which of the top or bottom sides 312, 315 the adhesive is located. Thus, both anchor sheets 310a, 310b can be applied to the skin of the patient in a direction toward the access site 108, or away from the access site 108. In another embodiment, the adhesive is applied to the bottom side 315 of one anchor sheet and to the top side of one anchor sheet, such that one anchor sheet 310a is applied to the skin of the patient in one direction, and the other anchor sheet 310b is applied to the skin of the patient in the other opposite direction.

Methods of making and of using the catheter assemblies with securing mechanisms and their components as discussed elsewhere herein are within the scope of the present invention.

Although limited embodiments of various catheter assemblies having a securing mechanism of the anchor sheets have been specifically described and illustrated herein, many modifications and variations will be apparent to those skilled in the art. For example, any over the catheter assemblies can benefit by using the securing mechanism disclosed herein to provide quicker attachment to the patient. Furthermore, it is understood and contemplated that features specifically discussed for one catheter assemblies with securing mechanisms may be adopted for inclusion with another catheter assembly provided the functions are compatible. Accordingly, it is to be understood that the catheter assemblies with securing mechanisms and their components constructed according to principles of the disclosed devices, systems, and methods may be embodied other than as specifically described herein. The disclosure is also defined in the following claims.

1. A catheter assembly with securing mechanisms, the catheter assembly comprising:
a catheter hub unit comprising a catheter hub, a catheter tube extending distally from the catheter hub, and two wings each extending laterally of the catheter hub;
a needle hub unit comprising a needle hub and a needle extending distally from the catheter hub and projecting through the catheter tube in a ready position;
an anchor sheet located on each of the wings, each anchor sheet comprising a top side facing away from a corresponding wing and a bottom side facing the corresponding wing in the ready position;
an adhesive layer provided on the top side of each anchor sheet;
a release liner covering the adhesive layer on each anchor sheet in the ready position; and
wherein each anchor sheet is pivotably connected to the corresponding wing.

2. The catheter assembly of claim 1, wherein each anchor sheet is pivotably connected to the corresponding wing in a different direction from each other.

3. The catheter assembly of claim 1, wherein each wing is shaped differently from the other wing.

4. The catheter assembly of claim 1, wherein a recessed space is formed in each wing, the recessed space being configured for securing the anchor sheet in the ready position.

5. The catheter assembly of claim 4, wherein a tab extends from each anchor sheet, the tab being configured for pulling the anchor sheet from the recessed space.

6. The catheter assembly of claim 5, wherein a release liner tab extends from the release liner.

7. The catheter assembly of claim 1, wherein each anchor sheet is pivotably connected to the respective wing by adhesive along a pivoting edge on the anchor sheet.

8. The catheter assembly of claim 1, wherein each anchor sheet is pivotably connected to the respective wing by a living hinge.

9. The catheter assembly of claim 8, wherein the living hinge is located at a proximal end of the wing.

10. The catheter assembly of claim 9, wherein the release liner is attached to the needle hub unit, withdrawing the needle hub unit from the catheter hub unit rotates the anchor sheet and removes the release liner.

11. The catheter assembly of claim 1, wherein the anchor sheets comprises a plurality of connected subsections folded upon each other in an accordion fashion to form folded anchor sheets.

12. The catheter assembly of claim 11, wherein the needle hub unit further comprises a pair of tethers, the tethers are attached to a free end of the folded anchor sheets.

13. The catheter assembly of claim 12, wherein the folded anchor sheets are stored in a storage space in the wing.

14. The catheter assembly of claim 12, wherein the folded anchor sheets are tucked under the wing.

15. The catheter assembly of claim 12, wherein the folded anchor sheets unfold to extend in a same first direction.

16. The catheter assembly of claim 14, wherein one of the two folded anchor sheets unfold to extend in a distal direction with the adhesive facing down, and the other of the two anchor sheets unfold to extend in a proximal direction with the adhesive facing down.

17. A method of manufacturing a catheter assembly, the method comprising:
extending a catheter tube distally from a catheter hub, a left wing laterally from a left side of the catheter hub, and a right wing laterally from a right side of the catheter hub;
extending a needle from a needle hub unit through the catheter tube in a ready position;
pivotably connecting an anchor sheet to the left wing and another anchor sheet to the right wing, each anchor sheet comprising a top side facing away from a corresponding wing and a bottom side facing the corresponding wing in the ready position, the top side having an adhesive layer; and
covering the adhesive layer on each anchor sheet with a release liner.

18. The method of claim 17, wherein each anchor sheet is pivotably connected to the corresponding wing in a different direction from each other.

19. The method of claim 17, further comprising forming a recessed space in each wing, the recessed space securing the anchor sheet in the ready position.

20. The method of claim 19, further comprising pulling the anchor sheet from the recessed space.

21. The method of claim 17, wherein each anchor sheet is pivotably connected to the corresponding wing by a living hinge.

22. The method of claim 21, wherein the living hinge is located at a proximal end of the wing.

23. The method of claim 22, further comprising withdrawing the needle hub from the catheter hub thereby rotating the anchor sheet and removing the release liner, wherein the release liner is attached to the needle hub.

24. The method of claim 17, further comprising folding the anchor sheets into a plurality of connected subsections folded upon each other in an accordion fashion.

25. The method of claim 24, further comprising unfolding the anchor sheets by pulling a free end of the folded anchor sheets with tethers attached to the needle hub.

26. The method of claim 25, further comprising storing the anchor sheets in a storage space in the wing.

27. The method of claim 25, further comprising tucking the folded anchor sheets under the wing.

28. The method of claim 25, further comprising folding the anchor sheets over the wings towards a distal direction after unfolding the anchor sheets.

29. The method of claim 25, further comprising unfolding the anchor sheets in different directions.
